(19) United States
(12) Patent Application Publication
(10) Pub. No.: US 2004/0010129 A1
(43) Pub. Date: Jan. 15, 2004

(54) Nucleic acid kit for bacterial pathogen diagnosis and method for using the same

(76) Inventors: Po-Hsiang Hsu, Taipei City (TW); Ya-Lin Chiang, Taipei City (TW); Hsien Long Huang, Taipei City (TW); Shou Yu Chao, Taipei City (TW)

Correspondence Address: WILDMAN, HARROLD, ALLEN & DIXON, 225 WEST WACKER DRIVE, CHICAGO, IL, 60606, US

(21) Appl. No.: 10/281,845

(22) Filed: Oct. 28, 2002

(30) Foreign Application Priority Data
Nov. 1, 2001 (CN) 090127119

(51) Int. Cl. C07H 21/02; C07H 21/04

(52) U.S. Cl. 536/23.1

(57) ABSTRACT

The present invention relates to a nucleic acid kit for bacterial pathogen diagnosis and method for using the same, which provides with a quick diagnosis for 20 species of bacterial pathogens. The present invention is to align the nucleic acid sequences of each bacterial pathogen, single out the specific region thereof, find out the corresponsive consensus primers, and amplify the specific nucleic acid sequences of each bacterial pathogen, whereafter the nucleic acid kit is acquired. Further, such nucleic acid kit can be utilized as probes to be conjugated on polymers as diagnostic chips for bacterial pathogens (for example, the meningitis chip), and then the detection reaction proceeds as the nucleic acid sequences of pathogen purified from clinical sample and amplified by using the foregoing primers are to react with the bacterial pathogen diagnostic chip of the present invention, with the species of the infecting bacteria thus being determined.

### BACKGROUND OF THE INVENTION

### DESCRIPTION OF RELATED ARTS

Infectious diseases are often caused by pathogenic microbes or harmful byproducts produced therefrom, with such pathogenic microbes including bacteria, viruses, fungi and Rickettsia, wherein bacterial infectious diseases are the most common. Most infectious diseases caused by bacterial pathogens are not fatal, yet some of them still pose serious threat, such as pneumonia, meningitis and dysentery. Traditional diagnosis regarding bacterial pathogens involves collecting a patient's clinical sample, culturing bacteria therein, bio-chemically analyze bacteria cultured, and ascertaining the species of bacteria pathogen, thus antibiotics specifically for eliminating such species of bacteria can be administered to such patient. However, the aforementioned diagnosis takes several days to acquire the outcome, thus often causing delay in diagnosis and missing the best timing for curing diseases. For example, meningitis, a legally notifiable infectious disease, is caused by bacterial pathogens such as viruses, bacteria and fungi that invade arachnoid, meninges and cerebrospinal fluid. Thus any kind of bacteria may be possible to cause bacterial meningitis. Yet even though numerous bacteria may cause meningitis, 70% to 80% of meningitis cases are caused by Neisseria meningitides, Streptococcus pneumoniae and Haemophilus influenzae. Meningitis often are rampant in crowded environment such as schools or military barracks, with spring (March to June) being the major spreading season. Lumbar puncture is one of the most important examinations for diagnosing meningitis. As the central nerve system is infected with diseases causing apparent pathological variations, either the computerized axial tomography (CAT) scan or nuclear magnetic resonance (NMR) scan can be utilized for non-invasive scanning so as to diagnose the exact disease infected. However, provided a central nerve system is infected with certain infectious diseases causing no apparent pathological variations, such as meningitis, the lumbar puncture is then necessary to acquire cerebrospinal fluid for examination, thus accurate diagnosis can be obtained.

Infected pathogens cultured directly from cerebrospinal fluid is one crucial item necessary for accurate diagnosis, yet direct culturing of bacteria takes at least three days to over a week to complete, whereas antibody recognition, mostly being conducted in one-on-one pattern for bacteria recognition, requires higher cost thus causes waste of medical resources.

In view of the foregoing drawbacks existed in diagnosing meningitis pathogens, it can be concluded that higher cost and prolonged period for examination cause speedy and accurate diagnosis of meningitis pathogens to be unattainable. Therefore, a highly effective method and a kit for diagnosing meningitis pathogens is desperately needed for overcoming the foregoing drawbacks and raising the medical standard and efficiency.

### SUMMARY OF THE INVENTION

One of the objects in the present invention is to provide a nucleic acid kit for bacterial pathogen diagnosis, comprising one or more nucleic acid sequences that are designed for one or more bacterial pathogens, the foregoing pathogens are chosen from the next 20 pathogens: Staphylococcus aureus, Staphylococcus epidermidis, Streptococcus saprophyticus, Streptococcus agalactiae, Streptococcus pyogens, Streptococcus pneumoniae, Enterococcus faecium, Enterococcus faecalis, Mycobacterium tuberculosis, Legionella pneumophilia, Listeria monocytogene, Escherichia. coli, Klebsiella pneumoniae, Serratia marcescens, Enterobacter cloacae, Pseudomonas aeruginosa, Stenotrophomonas maltophilia, Proteus mirabilis, Haemophilus influenzae, Neisseria meningitides, and then the consensus sequence among the sub-species of pathogens (or variants and serotypes thereof) can be found out, which contains low or no homology to other meningitis pathogen genomic sequences apart from the pathogen itself. Therefore, the nucleic acid kit of the present invention designed by basing upon those consensus sequences can be widely applied to examinations on diseases caused by various bacterial pathogens. The nucleic acid kit can further be utilized to conjugate on a substrate as probes for detecting various kinds of bacterial pathogens, for example, when detecting the meningitis pathogen, the nucleic acid from a meningitis patient's clinical sample is to be hybridized, and then the foregoing nucleic acid kit conjugated on a substrate can be utilized here to detect the kind of pathogens causing meningitis with sensitivity and specificity. The foregoing substrate can be a biochip.

The other object of the present invention is to provide a chip utilized for diagnosing bacterial pathogens, comprising a substrate and one or more probes, which are chosen from nucleic acid sequences in the nucleic acid kit of the foregoing 20 pathogens, with the probes conjugated on the substrate. The chip can be utilized for detecting meningitis.

The substrate can be glass, nitrocellulose membrane, nylon membrane, silicon chip or polymers.

Another object of the present invention is to provide a method for detecting the nucleic acid of bacterial pathogens, comprising procedures of extracting the nucleic acid of specific bacterial pathogen, amplifying and labeling the extracted nucleic acid, hybridizing the amplified and labeled nucleic acid with the nucleic acid kit of the present invention, and eventually detecting signals generated after hybridization.

### BRIEF DESCRIPTION OF DRAWINGS

These and other features, aspects and advantages of the present invention will become better understood with regard to the following description, appended claims and accompanying drawings that are provided only for further elaboration without limiting or restricting the present invention, where:

FIG. 1 shows a result of implemented diagnosis by utilizing the chip in the embodiment of the present invention for diagnosing meningitis pathogens; and

FIG. 2 shows another result of implemented diagnosis by utilizing the chip in the embodiment of the present invention for diagnosing meningitis pathogens.

### DETAILED DESCRIPTION OF THE PREFERRED EMBODIMENTS

The following is a detailed description of the best presently known modes of carrying out the inventions. This description is not to be taken in a limiting sense, but is made merely for the purpose of illustrating the general principles of the inventions.

Regarding traditional medical detections, the culturing of bacteria is often time-consuming with recognition efficiency being less than satisfactory. In view of the superior qualities of biochips that provide users with speedy detection, simple operation and lower cost, thus improving the efficiency tremendously and corresponding to the market needs, the present invention, regarding the diagnosing procedure for bacterial pathogens, hereby provides with a nucleic acid kit and applied chip thereof (such as chips for diagnosing meningitis) for diagnosing and identifying pathogens, so as to improve upon the quality of medical detection regarding bacterial pathogens.

In the fabricating procedure of the nucleic acid kit of the present invention for diagnosing bacterial pathogens, twenty common bacteria causing meningitis are chosen to be the subject matters of the detection, with the species of sen listed as follows:

| | | Number of the |
|---|---|---|
| | | consensus sequences |
| | | provided by present |
| No. | Bacteria species | invention |
| 1 | Staphylococcus aureus | 1-1, 1-2 and 1-3 |
| 2 | Staphylococcus epidermidis | 2-1, 2-2 and 2-3 |
| 3 | Staphylococcus saprophyticus | 3-1, 3-2 and 3-3 |
| 4 | Streptococcus agalactiae | 4-1, 4-2 and 4-3 |
| 5 | Streptococcus. pyogens | 5-1, 5-2 and 5-3 |
| 6 | Streptococcus pneumoniae | 6-1, 6-2 and 6-3 |
| 7 | Enterococcus faecium | 7-1, 7-2 and 7-3 |
| 8 | Enterococcus faecalis | 8-1, 8-2 and 8-3 |
| 9 | Mycobacterium tuberculosis | 9-1, 9-2 and 9-3 |
| 10 | Legionella pneumophilia | 10-1, 10-2 and 10-3 |
| 11 | Listeria monocytogenes | 11-1, 11-2 and 11-3 |
| 12 | Escherichia. coli | 12-1, 12-2 and 12-3 |
| 13 | Klebsiella pneumoniae | 13-1, 13-2 and 13-3 |
| 14 | Serratia marcescens | 14-1, 14-2 and 14-3 |
| 15 | Enterobactercloacae | 15-1, 15-2 and 15-3 |
| 16 | Pseudomonas aeruginosa | 16-1, 16-2 and 16-3 |
| 17 | Stenotrophomonas maltophilia | 17-1, 17-2 and 17-3 |
| 18 | Proteus mirabilis | 18-1, 18-2 and 18-3 |
| 19 | Haemophilus influenzae | 19-1, 19-2 and 19-3 |
| 20 | Neisseria meningitidis | 20-1, 20-2 and 20-3 |

The consensus primers for the 20 bacterial pathogens are listed as follows:

| Primer | | |
|---|---|---|
| Name\: | Sequence 5′to 3′ | Bases |
| F | 5′-GAAGAGTTTGATCMTGGCTC-3′ | 20 |
| | (M = A + C) | |
| R | 5′-ACTGCTGCCTCCCGTAGGAG-3′ | 20 |

Furthermore, for each of the 20 pathogen nucleic acid sequences, three hybridized nucleic acid fragments that are complementary and can be utilized for diagnosis basis are determined respectively as a diagnostic group for the nucleic acid kit.

```
  1-1
CGGACGAGAAGCTTGCTTCTCTGATGTTAGCG
  1-2
TTTGAACCGCATGGTTCAAAAGTGAAAGACGG
  1-3
TTGCTGTCACTTATAGATGGATCCGCGCTGC
  2-1
AACAGACGAGGAGCTTGCTCCTCTGACGTTAGC
  2-2
GGATAATATATTGAACCGCATGGTTCAATAGTGAAAGACGC
  2-3
GTGAAAGACGGTTTTGCTGTCACTTATAGATGGATCCG
  3-1
TAAGGAGCTTGCTCCTTTGACGTTAGCGGC
  3-2
CATTTGGACCCGCATGGTTCTAAAGTGAAAGATG
  3-3
ATGGTTTTGCTATCACTTATAGATGGACCCGCGC
  4-1
CTGAGGTTTGGTGTTTACACTAGACTGATGAGTTGCGA
  4-2
GTAATTAACACATGTTGGTTATTTAAAAGGAGCAATTGCTTCACTG
  4-3
GGTTATTTAAAAGGAGCAATTGCTTCACTGTGAGATGGAC
  5-1
CTGAGAACTGGTGCTTGCACCGGTTCAAGG
  5-2
AAGAGAGACTAACGCATGTTAGTAATTTAAAAGGGGCAA
  5-3
GCATGTTAGTAATTTAAAAGGGGCAATTGCTCCACTATG
  6-1
AGAACGCTGAAGGAGGAGCTTGCTTCTCTGGAT
  6-2
AAGAGTGGATGTTGCATGACATTTGCTTAAAAGGTGC
  6-3
GACATTTGCTTAAAAGGTGCACTTGCATCACTACCAG
  7-1
CTTTTTCCACCGGAGCTTGCTCCACCGGAAA
  7-2
TATAACAATCGAAACCGCATGGTTTTGATTTGAAAGG
  7-3
TTGATTTGAAAGGCGCTTTCGGGTGTCG
  8-1
TCTTTCCTCCCGAGTGCTTGCACTCAATTGG
  8-2
CAGTTTATGCCGCATGGCATAAGAGTGAAAGGC
  8-3
TTCGGGTGTCGCTGATGGATGGACCCG
  9-1
GAAAGGTCTCTTCGGAGATACTCGAGTGGCGAAC
  9-2
GGACCACGGGATGCATGTCTTGTGGTG
  9-3
TCTTGTGGTGGAAAGCGCTTTAGCGGTGTG
10-1
GCAGCATTGTCTAGCTTGCTAGACAGATGGCGA
10-2
ATGTCTGAGGACGAAAGCTGGGGACCTTCG
10-3
CTGGGGACCTTCGGGCCTGGCGCTTTAAGATTA
11-1
AACGGAGGAAGAGCTTGCTCTTCCAAAGTTAGTGG
11-2
AATGATAAAGTGTGGCGCATGCCACGCTTT
11-3
CCACGCTTTTGAAAGATGGTTTCGGCTATCG
12-1
CAGGAAGCAGCTTGCTGCTTTGCTGACG
12-2
ACGTCGCAAGACCAAAGAGGGGGACCTTC
12-3
GGGCCTCTTGCCATCGGATGTGCC
13-1
GCGGTAGCACAGAGAGCTTGCTCTCGGG
13-2
TGTCGCAAGACCAAAGTGGGGGACCTTC
13-3
CAAAGTGGGGGACCTTCGGGCCTCAT
14-1
AGGACAGGGGAGCTTGCTCCCTGGGT
14-2
AACGTCGCAAGACCAAAGAGGGGGACCTTC
14-3
GAAAGAGGGGGACCTTCGGGCCTCTTG
15-1
GTAACAGGAAGCAGCTTGCTGCTTCGCTGAC
15-2
CGTCGCAAGACCAAAGAGGGGGACCTTC
15-3
CTTGCCATCGGATGTGCCCAGATGGG
16-1
GAAGGGAGCTTGCTCCTGGATTCAGCGG
16-2
GTCCTGAGGGAGAAAGTGGGGGATCTTCGG
16-3
TTCGGACCTCACGCTATCAGATGAGCCTAGGTC
17-1
GCAGCACAGGAGAGCTTGCTCTCTGGGTG
17-2
ACTTTTTCGTGGGGGATAACGTAGGGAAACTTACG
17-3
CGACCTACGGGTGAAAGCAGGGGATCTTC
18-1
GCGGTAACAGGAGAAAGCTTGCTTTCTTGCTGA
18-2
CCGATAGAGGGGGATAACTACTGGAAACGGTGG
18-3
GCTCTTCGGACCTTGCACTATCGGATGAACC
19-1
GTAGCAGGAGGAAGCTTGCTTTCTTGCTGACG
19-2
CGAGAGACGAAAGTGCGGGACTGTAAGGCC
19-3
CGCATGCCATAGGATGAGCCCAAGTGG
20-1
GCAGCACAGAGAAGCTTGCTTCTCGGGTG
20-2
CGTCTTGAGAGAGAAAGCAGGGGACCTTCGG
20-3
CTTGCGCTATTCGAGCGGCCGATATCTG
```

Each foregoing group of nucleic acid sequences for diagnosing pathogens includes at least one, two or three kinds of specific hybridized nucleic acid sequences. The nucleic acid sequences in the nucleic acid kit of the present invention can be individually utilized or utilized in accordance with other nucleic acid kits without being limited.

The nucleic acid kit of the present invention can further be conjugated on a substrate as probes, for example, at least one probe of the bacterial pathogen is to be planted on a proper substrate, with each probe containing different sequences complementary to a portion of the sequences in the nucleic acid of the pathogen targeted for detection. Probes can be conjugated on a substrate through in situ or ex situ synthesis, wherein the common in situ synthesis provides with spotting, ink-jetting or piezoelectric means for directly conjugating the synthesized probes on a substrate, whereas the ex situ synthesis provides with synthesizing probe sequences directly on a solid substrate. The substrate in the foregoing synthesizing methods can be made of nylon membrane, glass or polymer.

In addition, the present invention provides with the design of planting different kinds of probes respectively on different areas of a substrate, so as to identify the kinds of pathogens, whereby numerous non target probes are planted in areas outside a small area planted with target probes, or the area of the substrate can be divided into numerous specific target areas for planting various detecting probes, so as to detect different targets simultaneously. The other object of the present invention is to provide chips for diagnosing bacterial pathogens, wherein the preferred embodiment thereof is chips for diagnosing meningitis, with the procedure being that specific probes from 20 species of meningitis pathogen are to be planted in one chip, so as to identify the of meningitis pathogen for accurate diagnosis and administering of medicine.

The substrate can be made of glass, nitrocellulose membrane, nylon membrane, silicon chip or polymer. The conventional means of planting probes on substrates can be employed, with the targets or probes being adhered to solid substrates, so as to proceed to fragment hybridization with the supplementary nucleic acid of bacterial pathogens in the solution. The exemplary solid pattern includes Southern hybridization, blotting and similar means. The detection of hybridization can be preceded on solid substrates such as microplates, filtering membranes (i.e., nitrocellulose membranes), microspheres (tiny beads) chips or any feasible hybridization buffering fluid system.

Regarding the processing of bacterial pathogens, the nucleic acid in meningitis pathogens is to be extracted, then amplified with polymerase chain reaction (PCR) and marked, so as to enhance the binding force during hybridization with probes.

The characteristics and merits of the present invention can be apparently shown in the preferred embodiments and claims elaborated as follows.

Preferred Embodiments

The following is a description of the exemplary case of carrying out the diagnostic chip provided by the invention for diagnosing meningitis bacterial pathogens. This exemplary case is not to be taken in a limiting sense, but is made merely for the purpose of further illustrating the materials and methods for practicing the present invention. The chip for diagnosing meningitis bacterial pathogens of the present invention is carried out through the following steps:

A. Purification of Bacterial DNA:

(1) Take some bacteria from a colony and suspend in 500 μl solution I, which contains:

50 mM Glucose

10 mM EDTA

25 mM Tris-HCL, pH 8.0

(2) For Gram-positive bacteria, add 1 mg/ml lysozyme and incubate at 37° C. for 30-60 min. For Gram-negative bacteria, directly jump to step (3).

(3) Add 50 μl of 10% SDS and incubate at 65° C. for 30-60 min.

(4) Add 4 μl of RNase incubate at 37° C. for 30-60 min.

(5) Add 100 μl of 5M KAc and 300 μl of CHCl₃.

(6) Stir to mix for 15 sec and then centrifuge at 12000 rpm for 5 min.

(7) Transfer the supernatant to a new tube and add 2× volume of 95% ethanol.

(8) Mix well and centrifuge again.

(9) Decant the supernatant and wash the pellet with 70% ethanol.

(10) Decant the supernatant and air-dry the pellet.

(11) Dissolve the pellet with 500 μl of ddH₂O or TE buffer (TE buffer: mixture of 10 mM Tris-HCl and 1 mM EDTA, pH 8.0).

(12) Read OD260 of DNA solution in a spectrophotometer to determine the concentration of DNA.

(13) Dilute the DNA solution to concentration of 2 ng/ul for the following Polymerase Chain Reaction (PCR).

B. Polymerase Chain Reaction (PCR)

Prepare the Following Materials:

| | |
|---|---|
| (1) DNA template | 2λ |
| (2) 10X Reaction buffer (25 mM) | 5λ |
| (3) Forward Primer | 1λ |
| (4) Reverse Primer | 1λ |
| (5) Taq polymerase | 1U |
| (6) Digoxigenin(DIG)-dNTp | 2λ |

The reaction is carried out at 95° C. for 10 min to denature DNA, and continues with 30 cycles of reaction (95° C., 1 min; 58° C., 1 min; 72° C., 2 min), and at 72° C., 10 min at end to allow complete elongation of all product DNAs. Then, 3 μl of the PCR products are taken for the following chip hybridization.

C. Chip Hybridization

The procedures of testing meningitis diagnostic chip are as follows:

(1) Use 3 μl of the PCR product to react with the meningitis diagnostic chip.

(2) Carry out pre-hybridization (blocking), 30 min, and then add labeled probes for hybridization, 6 hrs.

(3) Wash away the unbound labeled probes, 3 hr.

(4) Scan the chip for signal detection, 20 min.

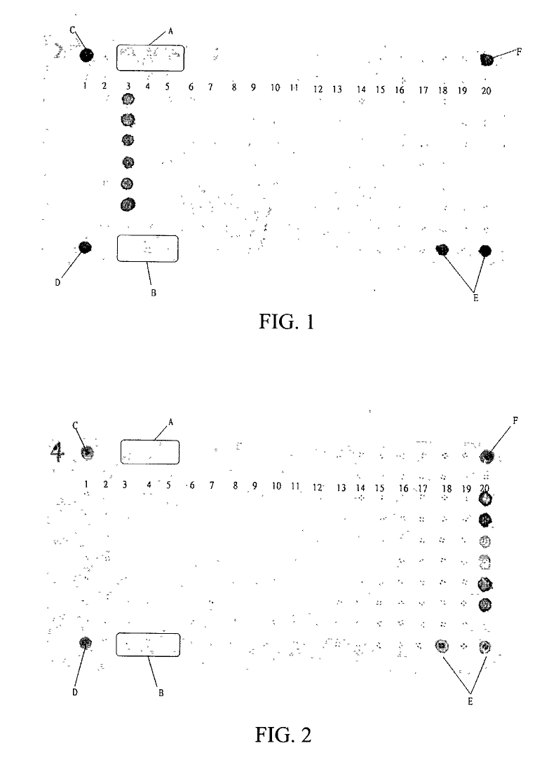

Afterwards, the signals read on meningitis diagnostic chip corresponded with the specific probe location are used for determining the species of the infectious pathogens. As shown in FIG. 1 and FIG. 2, three species of probes are adhered to the chip in this exemplary case: probes specifically to pathogens, probes for PCR positive control, probes for signal detection positive control. The main functions of these probes are describe as follows:

(1) Probes Specifically to Pathogens:

Probes for diagnosis of the 20 pathogenic bacteria are conjugated to the chip in turn according to the order of numbers from 1 to 20, as shown in FIGS. 1 and 2. From up to down, left to right on the chip, there are 3 probes for each bacteria, each probe in duplicate (6 spots total for each pathogen). Depending on the signal location, the species of infectious bacterial pathogens can thus be determined.

(2) Probes for PCR Positive Control:

Signals present in region A and region B, as shown in FIGS. 1 and 2 indicate a trusty PCR process.

(3) Probes for Signal Detection Positive Control:

Signals present in region C, D, E and F, as shown in FIGS. 1 and 2 indicate a trusty signal detection process.

In the present invention, the signal is visible to the naked eye and therefore the results can be directly observed without any assistant device. As shown in FIG. 1, the bacterial pathogen infected is Staphylococcus saprophyticus, and as for FIG. 2 is Neisseria meningitides.

Furthermore, other methods for signal detection and the relevant labeling technologies, such as fluorescence labeling, radioisotope labeling, chemical labeling, or spectrophotometers, can also be applied to the nucleic acid kit of the present invention.

The probes of the present invention for diagnosing meningitis pathogens may detect up to twenty species of bacteria that cover 80% to 90% of s of bacteria, whereby meningitis patients might be infected, including Neisseria meningitides that causes the currently prevalent epidemic meningitis. In addition, the present invention also integrates numerous biological technologies that specific probes can be found by utilizing biological information for fabricating meningitis diagnosis chips, so that what the user needs to do is to extract the DNA of the bacteria acquired from the clinical sample, amplify the extracted DNA through PCR, and hybridize such DNA with chips; thus, after the reaction, the species of infecting bacterium can be identified through the naked eye without utilizing any other identification systems. Comparing to the conventional means of one-on-one detection, the means of one-on-many detection provided by the chips of the present invention can detect and identify the species of infecting bacteria within twenty-four hours, along with at least half of the cost being saved. It is no doubt for patients that the chip technology applied contributes to the breakthrough in the field of medical detection.

Although the present invention has been described in considerable detail with reference to certain preferred embodiments thereof, those skilled in the art can easily understand that all kinds of alterations and changes can be made within the spirit and scope of the appended claims. Therefore, the spirit and scope of the appended claims should not be limited to the description of the preferred embodiments contained herein.

What is claimed is:

1. A nucleic acid kit for diagnosis of bacterial pathogens, comprising:
one or more nucleic acid sequences designed for one or more bacterial pathogens, wherein said bacterial pathogens are selected from the group including Staphylococcus aureus, Staphylococcus epidermidis, Streptococcus saprophyticus, Streptococcus agalactiae, Streptococcus. pyogens, Streptococcus. pneumoniae, Enterococcus faecium, Entercococcus faecalis, Mycobacterium tuberculosis, Legionella pneumophilia, Listeria monocytogenes, Escherichia. coli, Klebsiella pneumoniae, Serratia marcescens, Enterobacter cloacae, Pseudomonas aeruginosa, Stenotrophomonas maltophilia, Proteus mirabilis, Haemophilius influenzae and Neisseria meningitides.

2. A nucleic acid kit for diagnosis of bacterial pathogens as in claim 1, wherein said nucleic acid sequences for diagnosing Staphylococcus aureus are selected from the group including:
1-1
CGGACGAGAAGCTTGCTTCTCTGATGTTAGCG,
1-2
TTTGAACCGCATGGTTCAAAAGTGAAAGACGG and
1-3
TTGCTGTCACTTATAGATGGATCCGCGCTGC.

3. A nucleic acid kit for diagnosis of bacterial pathogens as in claim 1, wherein said nucleic acid sequences for diagnosing Staphylococcus epidermidis are selected from the group including:
2-1
AACAGACGAGGAGCTTGCTCCTCTGACGTTAGC,
2-2
GGATAATATATTGAACCGCATGGTTCAATAGTGAAAGACGG and
2-3
GTGAAAGACGGTTTTGCTGTCACTTATAGATGGATCCG

4. A nucleic acid kit for diagnosis of bacterial pathogens as in claim 1, wherein said nucleic acid sequences for diagnosing Streptococcus saprophyticus are selected from the group including:
3-1
TAAGGAGCTTGCTCCTTTGACGTTAGCGGC,
3-2
CATTTGGACCCGCATGGTTCTAAAGTGAAAGATG and
3-3
ATGGTTTTGCTATCACTTATAGATGGACCCGCGC.

5. A nucleic acid kit for diagnosis of bacterial pathogens as in claim 1, wherein said nucleic acid sequences for diagnosing Streptococcus agalactiae are selected from the group including:
4-1
CTGAGGTTTGGTGTTTACACTAGACTGATGAGTTGCGA,
4-2
GTAATTAACACATG1TGGTTATTTAAAAGGAGCAATTGCTTCACTG
and
4-3
GGTTATTTAAAAGGAGCAATTGCTTCACTGTGAGATGGAC.

6. A nucleic acid kit for diagnosis of bacterial pathogens as in claim 1, wherein said nucleic acid sequences for diagnosing Streptococcus. pyogens are selected from the group including:
5-1
CTGAGAACTGGTGCTTGCACCGGTTCAAGG
5-2
AAGAGAGACTAACGCATGTTAGTAATTTAAAAGGGGCAA and
5-3
GCATGTTAGTAATTTAAAAGGGGCAATTGCTCCACTATG.

7. A nucleic acid kit for diagnosis of bacterial pathogens as in claim 1, wherein said nucleic acid sequences for diagnosing Streptococcus. pneumoniae are selected from the group including:
6-1
AGAACGCTGAAGGAGGAGCTTGCTTCTCTGGAT,
6-2
AAGAGTGGATGTTGCATGACATTTGCTTAAAAGGTGC and
6-3
GACATTTGCTTAAAAGGTGCACTTGCATCACTACCAG

8. A nucleic acid kit for diagnosis of bacterial pathogens as in claim 1, wherein said nucleic acid sequences for diagnosing Enterococcus faecium are selected from the group including:
7-1
CTTTTTCCACCGGAGCTTGCTCCACCGGAAA
7-2
TATAACAATCGAAACCGCATGGTTTTGAAAGG and
7-3
TTGATTTGAAAGGCGCTTTCGGGTGTCG.

9. A nucleic acid kit for diagnosis of bacterial pathogens as in claim 1, wherein said nucleic acid sequences for diagnosing Entercococcus faecalis are selected from the group including:
8-1
TCTTTCCTCCCGAGTGCTTGCACTCAATTGG
8-2
CAGTTTATGCCGCATGGCATAAGAGTGAAAGGC and
8-3
TTCGGGTGTCGCTGATGGATGGACCCG

10. A nucleic acid kit for diagnosis of bacterial pathogens as in claim 1, wherein said nucleic acid sequences for diagnosing Mycobacterium tuberculosis are selected from the group including:
9-1
GAAAGGTCTCTTCGGAGATACTCGAGTGGCGAAC
9-2
GGACCACGGGATGCATGTCTTGTGGTG and
9-3
TCTTGTGGTGGAAAGCGCTTTAGCGGTGTG.

11. A nucleic acid kit for diagnosis of bacterial pathogens as in claim 1, wherein said nucleic acid sequences for diagnosing Legionella pneumophilia are selected from the group including:
10-1
GCAGCATTGTCTAGCTTGCTAGACAGATGGCGA,
10-2
ATGTCTGAGGACGAAAGCTGGGGACCTTCG and
10-3
CTGGGGACCTTCGGGCCTGGCGCTTTAAGATTA

12. A nucleic acid kit for diagnosis of bacterial pathogens as in claim 1, wherein said nucleic acid sequences for diagnosing Listeria monocytogenes are selected from the group including:
11-1
AACGGAGGAAGAGCTTGCTCTTCCAAAGTTAGTGG
11-2
AATGATAAAGTGTGGCGCATGCCACGCTTT and
11-3
CCACGCTTTTGAAAGATGGTTTCGGCTATCG.

13. A nucleic acid kit for diagnosis of bacterial pathogens as in claim 1, wherein said nucleic acid sequences for diagnosing Escherichia. coli are selected from the group including:
12-1
CAGGAAGCAGCTTGCTGCTTTGCTGACG
12-2
ACGTCGCAAGACCAAAGAGGGGGACCTTC and
12-3
GGGCCTCTTGCCATCGGATGTGCC.

14. A nucleic acid kit for diagnosis of bacterial pathogens as in claim 1, wherein said nucleic acid sequences for diagnosing Klebsiella pneumoniae are selected from the group including:
13-1
GCGGTAGCACAGAGAGCTTGCTCTCGGG
13-2
TGTCGCAAGACCAAAGTGGGGGACCTTC and
13-3
CAAAGTGGGGGACCTTCGGGCCTCAT.

15. A nucleic acid kit for diagnosis of bacterial pathogens as in claim 1, wherein said nucleic acid sequences for diagnosing Serratia marcescens are selected from the group including:
14-1
AGCACAGGGGAGCTTGCTCCCTGGGT
14-2
AACGTCGCAAGACCAAAGAGGGGGACCTTC and
14-3
CAAAGAGGGGGACCTTCGGGCCTCTTG.

16. A nucleic acid kit for diagnosis of bacterial pathogens as in claim 1, wherein said nucleic acid sequences for diagnosing Enterobacter cloacae are selected from the group including:
15-1
GTAACAGGAAGCAGCTTGCTGCTTCGCTGAC
15-2
CGTCGCAAGACCAAAGAGGGGGACCTTC and
15-3
CTTGCCATCGGATGTGCCCAGATGGG.

17. A nucleic acid kit for diagnosis of bacterial pathogens as in claim 1, wherein said nucleic acid fragments for diagnosing Pseudomonas aeruginosa are selected from the group including:
16-1
GAAGGGAGCTTGCTCCTGGATTCAGCGG
16-2
GTCCTGAGGGAGAAAGTGGGGGATCTTCGG and
16-3
TTCGGACCTCACGCTATCAGATGAGCCTAGGTC.

18. A nucleic acid kit for diagnosis of bacterial pathogens as in claim 1, wherein said nucleic acid sequences for diagnosing Stenotrophomonas maltophilia are selected from the group including:
17-1
GCAGCACAGGAGAGCTTGCTCTCTGGGTG
17-2
ACTTTTTCGTGGGGGATAACGTAGGGAAACTTACG and
17-3
CGACCTACGGGTGAAAGCAGGGGATCTTC.

19. A nucleic acid kit for diagnosis of bacterial pathogens as in claim 1, wherein said nucleic acid sequences for diagnosing Proteus mirabilis are selected from the group including:
18-1
GCGGTAACAGGAGAAAGCTTGCTTTCTTGCTGA
18-2
CCGATAGAGGGGGATAACTACTGGAAACGGTGG and
18-3
GCTCTTCGGACCTTGCACTATCGGATGAACC

20. A nucleic acid kit for diagnosis of bacterial pathogens as in claim 1, wherein said nucleic acid sequences for diagnosing Haemophilius influenzae are selected from the group including:
19-1
GTAGCAGGAGGAAGCTTGCTTTCTTGCTGACG
19-2
CGAGAGACGAAAGTGCGGGACTGTAAGGCC and
19-3
CGCATGCCATAGGATGAGCCCAAGTGG.

21. A nucleic acid kit for diagnosis of bacterial pathogens as in claim 1, wherein said nucleic acid sequences for diagnosing Neisseria meningitides are selected from the group including:
20-1
GCAGCACAGAGAAGCTTGCTTCTCGGGTG
20-2
CGTCTTGAGAGAGAAAGCAGGGGACCTTCGG and
20-3
CTTGCGCTATTCGAGCGGCCGATATCTG.

22. A nucleic acid kit for diagnosis of bacterial pathogens as in claim 1, wherein said nucleic acid kit can be used for diagnosis of bacterial meningitis pathogens.

23. A nucleic acid kit for diagnosis of bacterial pathogens as in claim 1, wherein said nucleic acid kit can be conjugated to a substrate and served as probes.

24. A nucleic acid kit for diagnosis of bacterial pathogens as in claim 23, wherein said substrate can be biochips.

25. A nucleic acid kit for diagnosis of bacterial pathogens as in claim 24, wherein said biochip can be made of glass, nitrocellulose membrane, nylon membrane, silicon and polymer.

26. A method for detecting the nucleic acid of bacterial pathogens, comprising:
extracting the nucleic acid of bacterial pathogens;
amplifying and labeling the extracted bacterial nucleic acid as needed;
hybridizing the amplified bacterial nucleic acid with said nucleic acid kit as in claim 1; and
detecting the signal after hybridization.

27. A method for detecting the nucleic acid of bacterial pathogens as in claim 26, wherein the means for detection comprise fluorescence labeling, radioisotope labeling, chemical labeling, or spectrophotometer.

28. A chip for diagnosis of bacterial pathogens, comprising:
a substrate; and
one or more probes which can be selected from said nucleic acid kit as in claim 1,
wherein said probes are conjugated to said substrate.
